(19) United States
(12) Patent Application Publication
(10) Pub. No.: US 2026/0026790 A1
(43) Pub. Date: Jan. 29, 2026

(54) MAGNETIC SENSING FOR ROBOTIC SURGERY

(71) Applicant: ForSight Robotics Ltd., Yokneam Illit (IL)

(72) Inventors: Ori BEN ZEEV, Ramat Hasharon (IL); Tal KORMAN, Tel Aviv (IL); Daniel GLOZMAN, Kfar Yona (IL)

(21) Appl. No.: 19/282,691

(22) Filed: Jul. 28, 2025

Related U.S. Application Data
(60) Provisional application No. 63/676,544, filed on Jul. 29, 2024.

(51) Int. Cl. A61B 17/00; A61B 34/30; A61F 9/007

(52) U.S. Cl. CPC A61B 17/00; A61B 34/30; A61B 2017/00039; A61B 2017/00477; A61F 9/007

(57) ABSTRACT

Apparatus and method are described for operating a tool and for use with a robotic arm. A tool holder is configured to hold the tool. An end effector is configured to mount onto the robotic arm and to couple to the tool holder while mounted onto the robotic arm. The end effector includes a lock, configured to transition between a locked state, in which the lock locks the tool holder to the end effector, and an unlocked state, in which the lock does not lock the tool holder to the end effector, and comprising a magnet that produces a magnetic field. A magnetic sensor is configured to output a signal indicative of changes in the magnetic field, as sensed by the magnetic sensor, resulting from the lock transitioning between the locked state and the unlocked state. Other applications are also described.

### CROSS-REFERENCE TO RELATED APPLICATIONS

The present application claims the benefit of U.S. Provisional Application No. 63/676,544 to Ben Zeev at al., filed Jul. 29, 2024, entitled “Magnetic Sensing for Robotic Surgery,” which is incorporated herein by reference.

### TECHNICAL FIELD

Applications of the present disclosure are related generally to the field of robotics, and specifically to robotic surgery.

### BACKGROUND

In some types of robotic surgery, one or more robotic arms manipulate surgical tools under the control of an operator.

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and the face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more (e.g., 2-3) incisions, typically including at least one larger incision having a three-planar form, are made in the cornea of the eye. The incisions are typically made using a specialized blade, which is called a keratome blade. Subsequently, another anesthetic, such as lidocaine, is injected into the anterior chamber of the eye via the corneal incisions. Following this step, the pupil is dilated, and a viscoelastic injection is applied via the corneal incisions. The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed, using one or more tools inserted via the corneal incisions. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incisions, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. When the phacoemulsification is complete, the remaining lens cortex (i.e., the outer layer of the lens) and viscoelastic material is aspirated from the capsule. During the phacoemulsification and the aspiration, aspirated fluids are typically replaced with irrigation of a balanced salt solution, in order to maintain fluid pressure in the anterior chamber.

In some cases, if deemed to be necessary, the capsule is polished. Subsequently, the intraocular lens (IOL) is inserted into the capsule. The IOL is typically foldable and is inserted in a folded configuration, before unfolding inside the capsule. If necessary, one or more of the incisions are sealed by elevating the pressure inside the bulbus oculi (i.e., the globe of the eye), causing the internal tissue to be pressed against the external tissue of the incisions, such as to force closed the incisions.

### SUMMARY

In some surgical robotic systems, a tool holder is coupled to an end effector, which is mounted onto a robotic arm. The tool holder is configured to hold a tool that includes multiple teeth arranged circumferentially around the tool. The tool holder comprises a gear configured to interface with the teeth such that, when driven by the end effector, the gear rotates the tool.

However, whereas the tool holder is typically sterile, the end effector is typically not. It is therefore challenging to design a gear system that maintains the sterility of the tool holder, yet allows easy coupling of the tool holder to the end effector.

To address this challenge, applications of the present disclosure provide the tool holder with two gears, which are coupled to opposite ends of a shaft. One of the gears interfaces with the teeth of the tool, while the other gear interfaces with a gear belonging to the end effector. A sterile barrier, comprising an elastomeric ring for example, surrounds the shaft between the two gears of the tool holder. Typically, by virtue of the sterile barrier, the end effector is configured for use, during a surgical procedure, without sterilization, and the tool holder remains sterile, during the surgical procedure, despite the end effector not being sterilized. Furthermore, typically, the tool holder is configured to couple easily to the end effector, via movement along a single axis.

In particular, applications of the present disclosure provide a system for rotating a tool that includes multiple teeth arranged circumferentially around the tool, the system being for use with a robotic arm. The system comprises an end effector, configured to mount onto the robotic arm and comprising an end-effector gear, and a tool holder, which is configured to couple to the end effector and is configured to hold the tool while coupled to the end effector. The tool holder comprises a first tool-holder gear, configured to interface with the end-effector gear, while the tool holder is coupled to the end effector, such that the end-effector gear rotates the first tool-holder gear. The tool holder further comprises a shaft coupled, at a first end of the shaft, to the first tool-holder gear such that the first tool-holder gear rotates the shaft. The tool holder further comprises a second tool-holder gear coupled to the shaft at a second end of the shaft such that the shaft rotates the second tool-holder gear, and configured to interface with the teeth, while the tool holder holds the tool, such that the second tool-holder gear rotates the tool. The tool holder further comprises a sterile barrier that surrounds the shaft between the second tool-holder gear and the first tool-holder gear.

In some applications, an elastomeric ribbon (e.g., an elastomeric ring) is positioned between the tool and the second tool-holder gear, and/or between the end-effector gear and the first tool-holder gear. Advantageously, the elastomeric ribbon generates backlash-reducing friction.

Applications of the present disclosure also facilitate ascertaining, automatically, whether the tool holder is coupled to the end effector and/or, if the tool holder is coupled to the end effector, whether the tool holder is locked in place. For example, in some applications, the tool holder comprises a magnet that produces a magnetic field, and the end effector comprises a magnetic sensor configured to sense the magnetic field when the tool holder is coupled to the end effector. Alternatively or additionally, the end effector comprises a lock configured to lock the tool holder to the end effector and comprising a magnet that produces a magnetic field. When the tool holder is locked to the end effector, the magnetic field is sensed with different properties, such as a different power and/or direction, by the magnetic sensor, relative to when the tool holder is unlocked.

In particular, applications of the present disclosure provide a system for operating a tool, the system being for use with a robotic arm. The system comprises a tool holder, configured to hold the tool, and an end effector configured to mount onto the robotic arm and to couple to the tool holder while mounted onto the robotic arm. The end effector comprises a magnetic sensor. In some applications, the tool holder comprises a magnet that produces a magnetic field, and the magnetic sensor is configured to output a signal in response to the magnetic field. Alternatively or additionally, the end effector comprises a lock configured to transition between a locked state, in which the lock locks the tool holder to the end effector, and an unlocked state, in which the lock does not lock the tool holder to the end effector. The lock comprises a magnet that produces a magnetic field, and the magnetic sensor is configured to output a signal indicative of changes in the magnetic field, as sensed by the magnetic sensor, resulting from the lock transitioning between the locked state and the unlocked state.

Alternatively or additionally to ascertaining whether the tool holder is coupled and/or locked to the end effector, the magnet of the tool holder can facilitate the automation of other functions. For example, in some applications, the magnet facilitates automatically identifying the type of tool that is held by the tool holder, and/or automatically tracking the rotation of the tool. Typically, in such applications, the tool holder comprises a tool support, which comprises the magnet, is configured to support the tool while the tool holder holds the tool, and is configured to undergo deflection at least as the tool is placed in the tool holder. The magnetic field produced by the magnet is sensed with different properties, such as a different power and/or direction, by the magnetic sensor, depending on the deflection.

In particular, some applications provide multiple tools of different types comprising respective circumferential portions, respective radii of which differ from each other at at least one range of angles. The tool support is configured to support any one of the tools at the circumferential portion of the tool, such that the deflection of the tool support is a function of the radius of the circumferential portion of the tool. Thus, the signal from the magnetic sensor indicates the radius, and hence the type, of the tool.

Alternatively or additionally, the circumferential portion of at least one tool has a non-constant radius. The tool support is configured to support the tool at the circumferential portion of the tool while the tool holder holds the tool, and to undergo deflection, by virtue of the non-constant radius, as the end effector rotates the tool with respect to the tool holder. Thus, the signal from the magnetic sensor varies as the tool is rotated, such that the signal facilitates tracking the rotation of the tool.

There is therefore provided, in accordance with some applications of the present disclosure, an apparatus for rotating a tool that includes multiple teeth arranged circumferentially around the tool, the apparatus being for use with a robotic arm. The apparatus includes an end effector, configured to mount onto the robotic arm and including an end-effector gear. The apparatus further includes a tool holder, which is configured to couple to the end effector and to hold the tool while coupled to the end effector. The tool holder includes a first tool-holder gear, configured to interface with the end-effector gear, while the tool holder is coupled to the end effector, such that the end-effector gear rotates the first tool-holder gear. The tool holder further includes a shaft coupled, at a first end of the shaft, to the first tool-holder gear such that the first tool-holder gear rotates the shaft. The tool holder further includes a second tool-holder gear coupled to the shaft at a second end of the shaft such that the shaft rotates the second tool-holder gear, and configured to interface with the teeth, while the tool holder holds the tool, such that the second tool-holder gear rotates the tool. The tool holder further includes a sterile barrier that surrounds the shaft between the second tool-holder gear and the first tool-holder gear.

In some applications, the sterile barrier includes an elastomeric ring.

In some applications, the tool holder is configured to couple to the end effector, such that the first tool-holder gear interfaces with the end-effector gear, via movement along a single axis.

In some applications, the end effector is shaped to define a cavity, and the tool holder is configured to couple to the end effector by fitting into the cavity.

In some applications, the end effector further includes a lock configured to lock the tool holder to the end effector while the tool holder is coupled to the end effector.

In some applications, the lock includes an arm configured to lock the tool holder to the end effector by rotating after passing through the tool holder.

In some applications, the tool holder is configured to couple to a sterile drape such that, when the tool holder is coupled to the end effector, the tool and the end effector are at opposite sides of the sterile drape.

In some applications, the tool includes a surgical tool.

In some applications, the surgical tool includes an ophthalmic surgical tool.

In some applications, by virtue of the sterile barrier:
the end effector is configured for use, during a surgical procedure, without sterilization, and
the tool holder remains sterile, during the surgical procedure, despite the end effector not being sterilized.

In some applications, the apparatus further includes an elastomeric ribbon positioned so as to interpose between the tool and the second tool-holder gear while the second tool-holder gear rotates the tool, thereby creating backlash-reducing friction between the tool and the second tool-holder gear.

In some applications, the apparatus further includes an elastomeric ribbon positioned so as to interpose between the end-effector gear and the first tool-holder gear while the end-effector gear rotates the first tool-holder gear, thereby creating backlash-reducing friction between the end-effector gear and the first tool-holder gear.

There is further provided, in accordance with some applications of the present disclosure, an apparatus for use with an end effector mounted onto a robotic arm. The apparatus includes a tool gear including multiple tool-gear teeth arranged circumferentially around the tool gear. The apparatus further includes a tool holder, which is configured to couple to the end effector, and which includes a tool-holder gear. The tool-holder gear includes multiple tool-holder-gear teeth arranged circumferentially around the tool-holder gear and is configured to interface with the tool gear, while the tool holder is coupled to the end effector, such that the robotic arm rotates the tool gear via the tool-holder gear. The apparatus further includes an elastomeric ribbon positioned so as to interpose between the tool gear and the tool-holder gear while the tool-holder gear interfaces with the tool gear, thereby creating backlash-reducing friction between the tool gear and the tool-holder gear.

In some applications, the elastomeric ribbon is ring-shaped.

In some applications, the apparatus further includes a tool including the tool gear, and the tool holder is further configured to hold the tool such that the tool-holder gear interfaces with the tool gear.

In some applications, the tool includes a surgical tool.

In some applications, the surgical tool includes an ophthalmic surgical tool.

In some applications,
the apparatus is for use with a tool,
the tool gear is configured to couple to the tool, and
the tool holder is further configured to hold the tool such that the tool-holder gear interfaces with the tool gear.

In some applications,
the tool-holder gear is shaped to define a groove passing through the tool-holder-gear teeth, and
the elastomeric ribbon is disposed within the groove such that the tool-gear teeth interface with the elastomeric ribbon while the tool-holder gear interfaces with the tool gear.

In some applications,
the groove is a first groove, and
the tool gear is shaped to define a second groove passing through the tool-gear teeth and positioned such that, while the tool-holder gear interfaces with the tool gear, the second groove is opposite the first groove.

In some applications, the groove passes through a center of the tool-holder-gear teeth.

In some applications, the elastomeric ribbon is coupled to the tool-holder gear adjacently to the tool-holder-gear teeth.

In some applications,
the tool gear is shaped to define a groove passing through the tool-gear teeth, and
the elastomeric ribbon is disposed within the groove such that the tool-holder-gear teeth interface with the elastomeric ribbon while the tool-holder gear interfaces with the tool gear.

In some applications,
the groove is a first groove, and
the tool-holder gear is shaped to define a second groove passing through the tool-holder-gear teeth and positioned such that, while the tool-holder gear interfaces with the tool gear, the second groove is opposite the first groove.

In some applications, the groove passes through a center of the tool-gear teeth.

In some applications, the elastomeric ribbon is coupled to the tool gear adjacently to the tool-gear teeth.

There is further provided, in accordance with some applications of the present disclosure, an apparatus for use with a robotic arm and a tool. The apparatus includes an end effector configured to mount onto the robotic arm and including an end-effector gear, which includes multiple end-effector teeth arranged circumferentially around the end-effector gear. The apparatus further includes a tool holder, which is configured to couple to the end effector and to hold the tool, and which includes a tool-holder gear. The tool-holder gear includes multiple tool-holder-gear teeth arranged circumferentially around the tool-holder gear and is configured to interface with the end-effector gear, while the tool holder is coupled to the end effector, such that the robotic arm rotates the tool via the end-effector gear and tool-holder gear. The apparatus further includes an elastomeric ribbon positioned so as to interpose between the end-effector gear and the tool-holder gear while the tool-holder gear interfaces with the end-effector gear, thereby creating backlash-reducing friction between the end-effector gear and the tool-holder gear.

In some applications, the elastomeric ribbon is ring-shaped.

In some applications, the tool includes a surgical tool.

In some applications, the surgical tool includes an ophthalmic surgical tool.

In some applications,
the tool-holder gear is shaped to define a groove passing through the tool-holder-gear teeth, and
the elastomeric ribbon is disposed within the groove such that the end-effector teeth interface with the elastomeric ribbon while the tool-holder gear interfaces with the end-effector gear.

In some applications,
the groove is a first groove, and
the end-effector gear is shaped to define a second groove passing through the end-effector-gear teeth and positioned such that, while the tool-holder gear interfaces with the end-effector gear, the second groove is opposite the first groove.

In some applications, the groove passes through a center of the tool-holder-gear teeth.

In some applications, the elastomeric ribbon is coupled to the tool-holder gear adjacently to the tool-holder-gear teeth.

In some applications,
the end-effector gear is shaped to define a groove passing through the end-effector-gear teeth, and
the elastomeric ribbon is disposed within the groove such that the tool-holder-gear teeth interface with the elastomeric ribbon while the tool-holder gear interfaces with the end-effector gear.

In some applications,
the groove is a first groove, and
the tool-holder gear is shaped to define a second groove passing through the tool-holder-gear teeth and positioned such that, while the tool-holder gear interfaces with the end-effector gear, the second groove is opposite the first groove.

In some applications, the groove passes through a center of the end-effector-gear teeth.

In some applications, the elastomeric ribbon is coupled to the end-effector gear adjacently to the end-effector-gear teeth.

In some applications, the tool-holder gear is a first tool-holder gear and the elastomeric ribbon is a first elastomeric ribbon, and the apparatus further includes:
a tool gear arranged circumferentially around the tool;
a shaft coupled, at a first end of the shaft, to the first tool-holder gear such that the first tool-holder gear rotates the shaft;
a second tool-holder gear coupled to the shaft at a second end of the shaft such that the shaft rotates the second tool-holder gear, and configured to interface with the tool gear, while the tool holder holds the tool, such that the second tool-holder gear rotates the tool; and
a second elastomeric ribbon positioned so as to interpose between the tool gear and the second tool-holder gear while the second tool-holder gear interfaces with the tool gear, thereby creating backlash-reducing friction between the tool gear and the second tool-holder gear.

There is further provided, in accordance with some applications of the present disclosure, an apparatus for operating a tool, the apparatus being for use with a robotic arm. The apparatus includes a tool holder, configured to hold the tool, and an end effector, configured to mount onto the robotic arm and to couple to the tool holder while mounted onto the robotic arm. The end effector includes a lock, configured to transition between a locked state, in which the lock locks the tool holder to the end effector, and an unlocked state, in which the lock does not lock the tool holder to the end effector, and including a magnet that produces a magnetic field. The end effector further includes a magnetic sensor, configured to output a signal indicative of changes in the magnetic field, as sensed by the magnetic sensor, resulting from the lock transitioning between the locked state and the unlocked state.

In some applications, the lock includes an arm configured to lock the tool holder to the end effector by rotating after passing through the tool holder.

In some applications, the tool includes a surgical tool.

In some applications, the surgical tool includes an ophthalmic surgical tool.

In some applications, the apparatus further includes a processor configured to:
receive the signal, and
process the signal so as to ascertain whether the lock is in the locked state.

In some applications,
the magnet is a first magnet and the magnetic field is a first magnetic field,
the tool holder includes a second magnet that produces a second magnetic field, and
the signal is indicative of the second magnetic field.

In some applications, the apparatus further includes a processor configured to:
receive the signal, and
process the signal so as to ascertain whether the tool holder is coupled to the end effector.

In some applications, the processor is further configured to ascertain whether the lock is in the locked state, by processing the signal.

In some applications, the processor is further configured to display an alert in response to ascertaining that the tool holder is coupled to, but not locked to, the end effector.

In some applications, the processor is further configured to refrain from controlling the robotic arm in response to ascertaining that the tool holder is coupled to, but not locked to, the end effector.

In some applications, the tool holder includes a tool support including the second magnet and configured to:
support the tool while the tool holder holds the tool, and
undergo deflection at least as the tool is placed in the tool holder such that the tool support supports the tool, the second magnetic field varying with the deflection.

In some applications, the apparatus further includes a processor configured to:
receive the signal, and
process the signal so as to ascertain a type of the tool.

In some applications, the tool support includes an arm configured to undergo deflection by pivoting.

In some applications,
the arm includes a roller configured to contact the tool while the tool holder holds the tool, and
the end effector is configured to rotate the tool against the roller.

In some applications,
the end effector is configured to rotate the tool, with respect to the tool holder, while the tool holder holds the tool,
the tool includes a circumferential portion having a non-constant radius, and
the tool support is configured to undergo deflection, by virtue of the non-constant radius, as the end effector rotates the tool.

In some applications, the apparatus further includes a processor configured to:
receive the signal, and
process the signal so as to track the rotation of the tool.

In some applications, the processor is configured to process the signal so as to track a roll orientation of the tool.

There is further provided, in accordance with some applications of the present disclosure, an apparatus for operating a tool, the apparatus being for use with a robotic arm. The apparatus includes a tool holder, configured to hold the tool and including a magnet that produces a magnetic field. The apparatus further includes an end effector, configured to mount onto the robotic arm and to couple to the tool holder while mounted onto the robotic arm, and including a magnetic sensor configured to output a signal in response to the magnetic field.

In some applications, the tool includes a surgical tool.

In some applications, the surgical tool includes an ophthalmic surgical tool.

In some applications, the apparatus further includes a processor configured to:
receive the signal, and
process the signal so as to ascertain whether the tool holder is coupled to the end effector.

In some applications, the tool holder includes a tool support including the magnet and configured to:
support the tool while the tool holder holds the tool, and
undergo deflection at least as the tool is placed in the tool holder such that the tool support supports the tool, the magnetic field varying with the deflection.

In some applications, the apparatus further includes a processor configured to:
receive the signal, and
process the signal so as to ascertain a type of the tool.

In some applications, the tool support includes an arm configured to undergo deflection by pivoting.

In some applications,
the arm includes a roller configured to contact the tool while the tool holder holds the tool, and
the end effector is configured to rotate the tool against the roller.

In some applications,
the end effector is configured to rotate the tool, with respect to the tool holder, while the tool holder holds the tool,
the tool includes a circumferential portion having a non-constant radius, and
the tool support is configured to undergo deflection, by virtue of the non-constant radius, as the end effector rotates the tool.

In some applications, the apparatus further includes a processor configured to:
receive the signal, and
process the signal so as to track the rotation of the tool.

In some applications, the processor is configured to process the signal so as to track a roll orientation of the tool.

In some applications,
the magnet is a first magnet and the magnetic field is a first magnetic field, and
the end effector further includes a lock configured to transition between a locked state, in which the lock locks the tool holder to the end effector, and an unlocked state, in which the lock does not lock the tool holder to the end effector,
the lock including a second magnet that produces a second magnetic field, and
the signal is indicative of changes in the second magnetic field, as sensed by the magnetic sensor, resulting from the lock transitioning between the locked state and the unlocked state.

In some applications, the lock includes an arm configured to lock the tool holder to the end effector by rotating after passing through the tool holder.

There is further provided, in accordance with some applications of the present disclosure, an apparatus for use with a magnetic sensor and an end effector mounted onto a robotic arm. The apparatus includes multiple tools of different types including respective circumferential portions, respective radii of which differ from each other at at least one range of angles, and a tool holder configured to hold any tool of the tools and to couple to the end effector so as to allow the end effector to rotate the tool with respect to the tool holder. The tool holder includes a tool support configured to support the tool at the circumferential portion of the tool while the tool holder holds the tool, and to undergo deflection at least as the tool is placed in the tool holder such that the tool support supports the tool, the deflection being a function of the radius of the circumferential portion of the tool. The tool support includes a magnet that produces a magnetic field that is sensed with different properties by the magnetic sensor, depending on the deflection.

In some applications, the tools include surgical tools.

In some applications, the surgical tools include ophthalmic surgical tools.

In some applications, the tool support includes an arm configured to undergo deflection by pivoting.

In some applications, the arm includes a roller configured to contact the tool while the tool holder holds the tool, such that the end effector rotates the tool against the roller.

In some applications, the radii differ from each other over an entirety of the circumferential portions.

In some applications, the magnetic sensor is configured to output a signal in response to the magnetic field, and the apparatus further includes a processor configured to:
receive the signal, and
process the signal so as to ascertain which of the tools is held by the tool holder.

In some applications, the radius of the circumferential portion of at least one of the tools is non-constant, such that the tool support undergoes deflection as the at least one of the tools rotates with respect to the tool holder.

In some applications, the respective radii differ from each other such that each of tools produces a unique pattern in the deflection, relative to others of the tools, when rotated by the end effector.

In some applications, the radius of the circumferential portion of the at least one of the tools at one range of angles is different from the radius at all angles outside the range.

In some applications, the radius of the circumferential portion of the at least one of the tools varies continuously between a minimum value and a maximum value, each of which is attained at a single respective angle.

There is further provided, in accordance with some applications of the present disclosure, an apparatus for use with a magnetic sensor and an end effector mounted onto a robotic arm. The apparatus includes a tool including a circumferential portion having a non-constant radius, and a tool holder configured to hold the tool and to couple to the end effector so as to allow the end effector to rotate the tool with respect to the tool holder. The tool holder includes a tool support configured to support the tool at the circumferential portion of the tool while the tool holder holds the tool, and to undergo deflection, by virtue of the non-constant radius, as the end effector rotates the tool with respect to the tool holder. The tool support includes a magnet that produces a magnetic field that is sensed with different properties by the magnetic sensor, depending on the deflection.

In some applications, the tool includes a surgical tool.

In some applications, the surgical tool includes an ophthalmic surgical tool.

In some applications, the tool support includes an arm configured to undergo deflection by pivoting.

In some applications, the arm includes a roller configured to contact the tool, while the tool holder holds the tool, such that the end effector rotates the tool against the roller.

In some applications, the radius of the circumferential portion of the tool at one range of angles is different from the radius at all angles outside the range.

In some applications, the radius of the circumferential portion of the tool varies continuously between a minimum value and a maximum value, each of which is attained at a single respective angle.

In some applications, the magnetic sensor is configured to output a signal in response to the magnetic field, and the apparatus further includes a processor configured to:
receive the signal, and
process the signal so as to track the rotation of the tool.

In some applications, the processor is configured to process the signal so as to track a roll orientation of the tool.

In some applications,
the radius of the circumferential portion of the tool at one range of angles is different from the radius at all angles outside the range, and
the processor is configured to track the roll orientation of the tool by:
   driving the end effector to rotate the tool to a reference orientation at which the deflection is different from at any other orientation of the tool, by virtue of the different radius, and
   tracking the roll orientation by tracking the rotation of the tool with respect to the reference orientation.

In some applications,
the radius of the circumferential portion of the tool varies continuously between a minimum value and a maximum value, each of which is attained at a single respective angle, and
the processor is configured to track the roll orientation of the tool based on the deflection and on a change in the deflection as the tool is rotated.

The present disclosure will be more fully understood from the following detailed description of applications thereof, taken together with the drawings, in which:

### BRIEF DESCRIPTION OF THE DRAWINGS

FIG. 1 is a schematic illustration of a robotic apparatus comprising one or more robotic arms, in accordance with some applications of the present disclosure;

FIGS. 2A and 2B are schematic illustrations of a tool-operating system configured to operate a tool, in accordance with some applications of the present disclosure;

FIG. 3A is a schematic illustration of a tool holder and an end portion of an end effector, in accordance with some applications of the present disclosure;

FIG. 3B shows a gear system belonging to a tool holder, and the end portion of an end effector with an outer wall of the end effector hidden from view, in accordance with some applications of the present disclosure;

FIG. 3C, 3D, and 3E are schematic illustrations showing the use of an elastomeric ribbon, in accordance with some applications of the present disclosure;

FIGS. 4A and 4B are schematic illustrations of a portion of an end effector to which a tool holder is coupled, in accordance with some applications of the present disclosure;

FIG. 5A is a schematic illustration of a tool holder, in accordance with some applications of the present disclosure;

FIG. 5B is a schematic illustration of a tool holder with a cover of the tool holder hidden from view, in accordance with some applications of the present disclosure;

FIGS. 6A and 6B illustrate the deflection of an arm for two tools of different types, in accordance with some applications of the present disclosure;

FIG. 7 is a schematic illustration of a circumferential portion of a tool, in accordance with some applications of the present disclosure;

FIGS. 8A and 8B schematically illustrate the rotation of a tool, in accordance with some applications of the present disclosure;

FIG. 9A is a schematic illustration of a circumferential portion of a tool 22, in accordance with some applications of the present disclosure; and

FIG. 9B plots the radius of the tool shown in FIG. 9A as a function of an angle, in accordance with some applications of the present disclosure.

### DETAILED DESCRIPTION

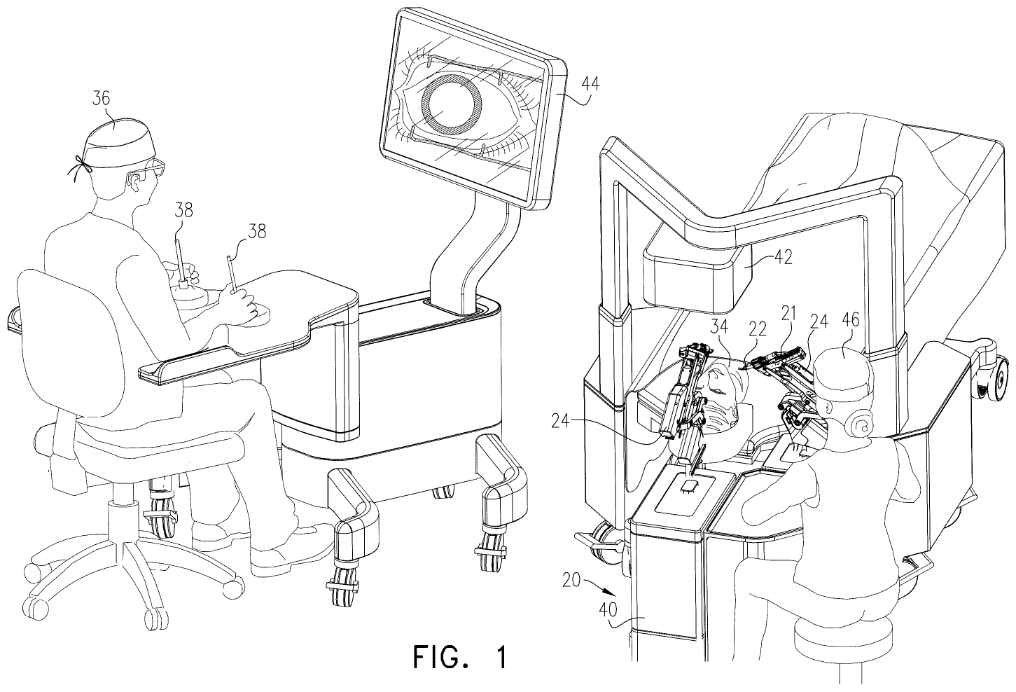

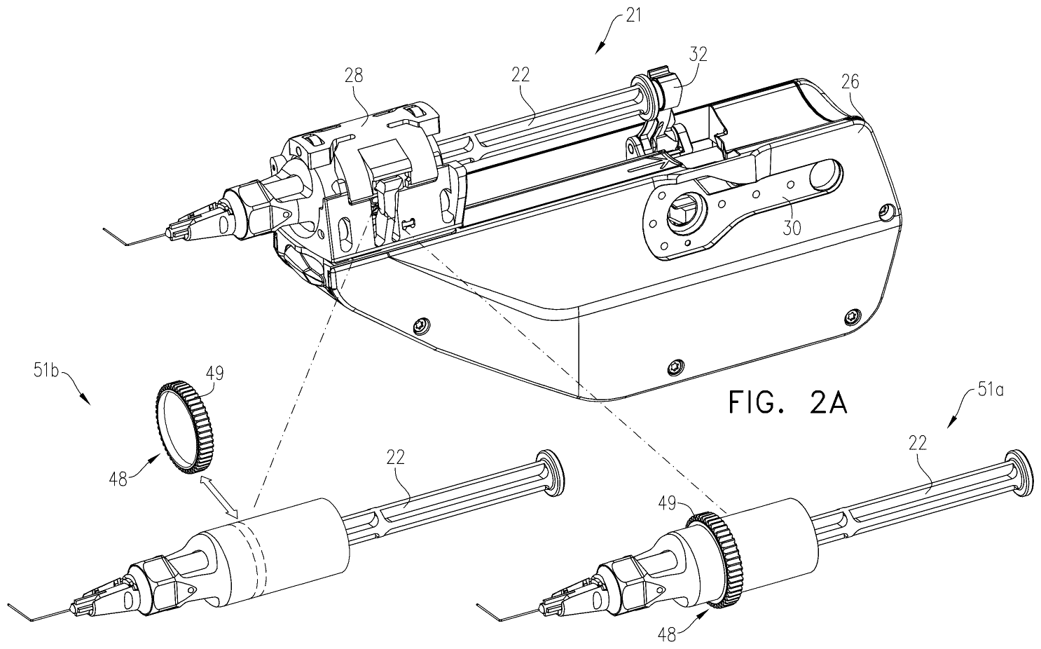

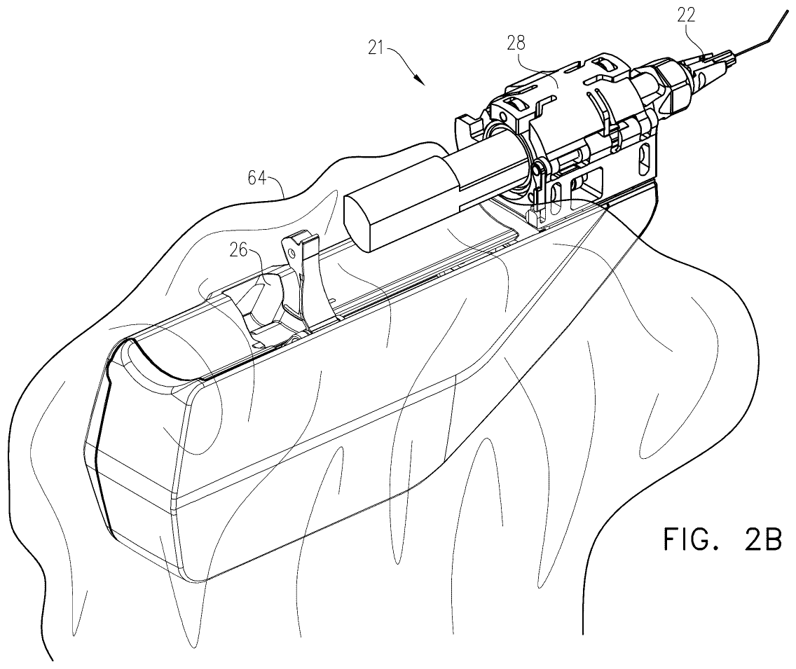

Reference is initially made to FIG. 1, which is a schematic illustration of a robotic apparatus 20 comprising one or more robotic arms 24, in accordance with some applications of the present disclosure. Reference is also made to FIG. 2A-B, which are schematic illustrations of a tool-operating system 21 configured to operate a tool 22, in accordance with some applications of the present disclosure.

Tool-operating system 21 comprises an end effector 26, which is configured to mount onto a robotic arm 24, e.g., via a slot 30 in end effector 26. Tool-operating system 21 further comprises a tool holder 28, which is configured to couple to end effector 26 and to hold tool 22 while coupled to the end effector. The robotic arm is configured to manipulate tool 22, via end effector 26, while tool holder 28 holds the tool. For example, typically, the robotic arm is configured to roll the tool, i.e., to rotate the tool about the axis of rotation of the tool. As another example, typically, end effector 26 comprises a pushing element 32, and the robotic arm is configured to push the tool, or a portion of the tool (e.g., the plunger of a syringe), using pushing element 32.

Typically, tool 22 includes a surgical tool, such as an ophthalmic surgical tool, e.g., a syringe, a phacoemulsifier, or a scalpel. For example, in some applications, apparatus 20 is configured to perform robotic surgery (e.g., ophthalmic surgery, such as cataract surgery) on a patient 34 using one or more tools 22. Apparatus 20 is controlled, remotely, by an operator 36, using one or more control tools 38 that correspond, respectively to robotic arms 24. In particular, as operator 36 manipulates control tools 38, a processor 40 drives the robotic arms to manipulate tools 22 in a corresponding manner. Typically, as operator 36 manipulates the control tools, the operator refers to images of the surgical site, which are acquired by an imaging system 42 and displayed on a display 44. During the procedure, operator 36 or an assistant 46 may load and unload tools from the tool-operating systems, as required for the procedure.

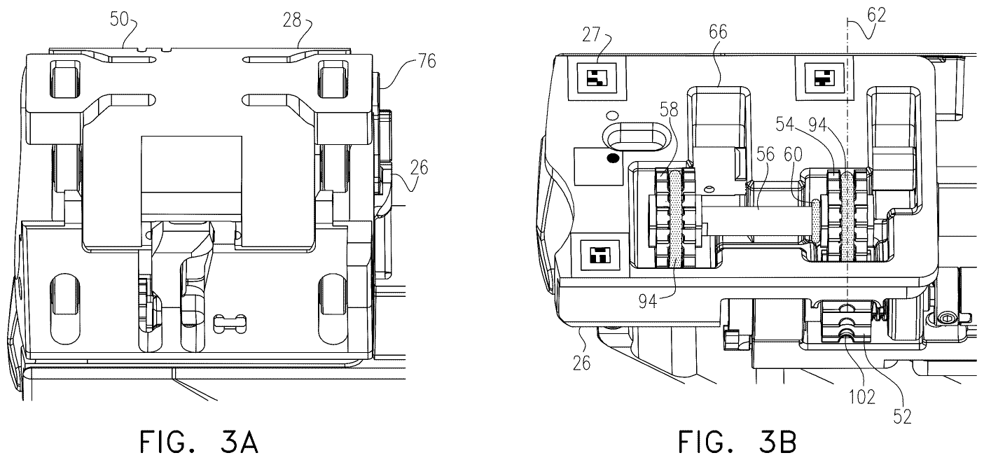

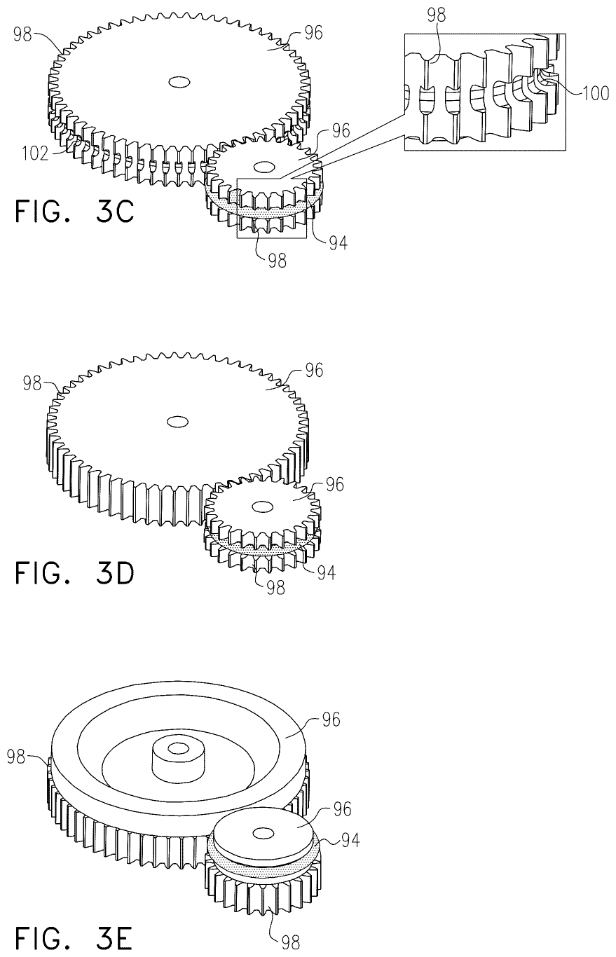

In some applications, as shown in FIG. 2A, the apparatus further comprises a tool gear 49, which comprises multiple teeth 48 are arranged circumferentially around tool gear 49. (Teeth 48 do not necessarily span 360 degrees.) In some applications, as shown in a first inset portion 51a of FIG. 2A, the tool comprises tool gear 49. In other applications, as shown in a second inset portion 51b, tool gear 49 is removably coupled to the tool during the use of the tool. As further explained below with reference to FIG. 3B, tool gear 49 facilitates rotating the tool.

Typically, as shown in FIG. 2B, tool holder 28 is configured to couple to a sterile drape 64 (e.g., via heat welding) such that, when the tool holder is coupled to end effector 26, tool 22 and end effector 26 are at opposite sides of sterile drape 64. Advantageously, by virtue of sterile drape 64, end effector 26 is configured for use, during a surgical procedure, without sterilization, and tool holder 28 remains sterile, during the surgical procedure, despite the end effector not being sterilized.

In general, processor 40 may be embodied as a single processor, or as a cooperatively networked or clustered set of processors. The functionality of processor 40 may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, processor 40 may be embodied as a programmed processor comprising, for example, a central processing unit (CPU) and/or a Graphics Processing Unit (GPU). Program code, including software programs, and/or data may be loaded for execution and processing by the CPU and/or GPU. The program code and/or data may be downloaded to the processor in electronic form, over a network, for example. Alternatively or additionally, the program code and/or data may be provided and/or stored on non-transitory tangible media, such as magnetic, optical, or electronic memory. Such program code and/or data, when provided to the processor, produce a machine or special-purpose computer, configured to perform the tasks described herein.

Reference is now made to FIG. 3A, which is a schematic illustration of tool holder 28 and an end portion of end effector 26 to which the tool holder is coupled, in accordance with some applications of the present disclosure.

Typically, tool holder 28 comprises a cover 50. Prior to inserting tool 22 (FIGS. 2A-B) into the tool holder, cover 50 is opened, e.g., by operating a release handle 76. After inserting the tool, the cover is closed.

Reference is now made to FIG. 3B, which shows a gear system belonging to tool holder 28, and the end portion of end effector 26 with the outer wall of the end effector hidden from view, in accordance with some applications of the present disclosure. In FIG. 3B, cover 50 and other components of the tool holder are hidden from view, so as to expose the gear system.

In some applications, tool holder 28 comprises a first gear 54 and a second gear 58, which are coupled to opposite ends of a shaft 56. Each of these gears comprises multiple teeth arranged circumferentially around the gear. (The teeth do not necessarily span 360 degrees).

End effector 26 comprises an end-effector gear 52. First gear 54 is configured to interface with end-effector gear 52, while the tool holder is coupled to the end effector, such that end-effector gear 52, when rotated by the robotic arm, rotates first gear 54. First gear 54, in turn, rotates shaft 56, which rotates second gear 58. Second gear 58 is configured to interface with tool gear 49 (FIG. 2A), while the tool holder holds tool 22, such that second gear 58 rotates the tool.

In general, each of the aforementioned gears may be of any suitable type. For example, in some applications, each gear comprises a spur gear. As another example, in some applications, end-effector gear 52 comprises a rack, and first gear 54 comprises a pinion.

Typically, tool holder 28 further comprises a sterile barrier 60, comprising an elastomeric ring (e.g., an O-ring) for example, that surrounds shaft 56 between first gear 54 and second gear 58. Typically, by virtue of sterile barrier 60, end effector 26 is configured for use, during a surgical procedure, without sterilization, and tool holder 28 remains sterile, during the surgical procedure, despite the end effector not being sterilized.

Typically, tool holder 28 is configured to couple to end effector 26, such that first gear 54 interfaces with end-effector gear 52, via movement along a single axis 62. For example, in some applications, the end effector is shaped to define a cavity 66, and the tool holder is configured to couple to the end effector by fitting into cavity 66. Thus, coupling the tool holder to the end effector is simplified, relative to if movement were required along multiple axes in order to align first gear 54 with end-effector gear 52.

In some applications, end effector 26 comprises registration markers 27, which facilitate registering the coordinate system of apparatus 20 with the coordinate system of imaging system 42 (FIG. 1).

In some applications, an elastomeric ribbon 94 is positioned so as to interpose between tool gear 49 (FIG. 2A) and second gear 58 while second gear 58 interfaces with the tool gear, thereby creating backlash-reducing friction between the tool gear and second gear 58 while second gear 58 rotates the tool. Alternatively or additionally, an elastomeric ribbon 94 is positioned so as to interpose between end-effector gear 52 and first gear 54 while these two gears interface with one another, thereby creating backlash-reducing friction between the two gears while end-effector gear 52 rotates first gear 54. Typically, elastomeric ribbon 94 is configured to reduce give between interlocking teeth belonging to the two gears, by providing friction between the two gear wheels even if there is a small gap between the interlocking teeth.

In some applications, elastomeric ribbon 94 is ring-shaped, e.g., elastomeric ribbon 94 comprises an O-ring. In other applications, elastomeric ribbon 94 is arced but spans less than 360 degrees.

For further details regarding the use of elastomeric ribbon 94, reference is now additionally made to FIGS. 3C-3E, which are schematic illustrations showing the use of elastomeric ribbon 94, in accordance with some applications of the present disclosure. Each of FIGS. 3C-3E shows a pair of gears 96, the teeth 98 of one gear interfacing with teeth 98 of the other gear. The pair of gears can represent second gear 58—or any other gear belonging to the tool holder in any suitable gear system—and tool gear 49 (FIG. 2A). Alternatively, the pair of gears can represent end-effector gear 52 and first gear 54. More generally, the pair of gears can belong to any gear system, which is not necessarily for use in robotic surgery.

In FIGS. 3C-3D, one of the gears is shaped to define a groove 100 passing through teeth 98, such as through the center of teeth 98, and elastomeric ribbon 94 is disposed within groove 100 such that the teeth of the other gear interface with the elastomeric ribbon. Thus, for example, assuming the gear with elastomeric ribbon 94 is rotating clockwise (such that the other gear is rotating counterclockwise), the elastomeric ribbon exerts a clockwise force on the teeth of the other gear, which reduces backlash when the direction of rotation is changed. FIGS. 3C and 3D differ from one another, however, in that in FIG. 3C, the other gear is shaped to define another groove 102 passing through teeth 98 and is positioned such that groove 102 is opposite groove 100. Advantages of groove 102 are less jerky rotation and less wear of elastomeric ribbon 94. On the other hand, an advantage of omitting groove 102, as shown in FIG. 3D, is greater case of manufacture. It is noted that the interface between end-effector gear 52 and first gear 54 shown in FIG. 3B corresponds to the interface in FIG. 3C, in that elastomeric ribbon 94 is disposed within a groove in first gear 54, and end-effector gear 52 is shaped to define an opposing groove 102.

In FIG. 3E, on the other hand, elastomeric ribbon 94 is coupled to one of the gears adjacently to the teeth of the gear. Thus, for example, assuming the gear with elastomeric ribbon 94 is rotating clockwise, the elastomeric ribbon exerts a clockwise force on the other gear, which reduces backlash when the direction of rotation is changed. Advantages of positioning the elastomeric ribbon adjacent to the teeth are less wear of the elastomeric ribbon and greater case of manufacture.

On the other hand, an advantage of positioning the elastomeric ribbon within groove 100 is that the groove helps hold the elastomeric ribbon in place.

As described hereinabove, elastomeric ribbon 94 is typically configured to reduce give between interlocking teeth belonging to the two gears, by providing friction between the two gear wheels even if there is a small gap between the interlocking teeth. For some applications, elastomeric ribbon 94 is configured to reduce backlash when the direction of rotation is changed and/or when rotation in a given direction is terminated.

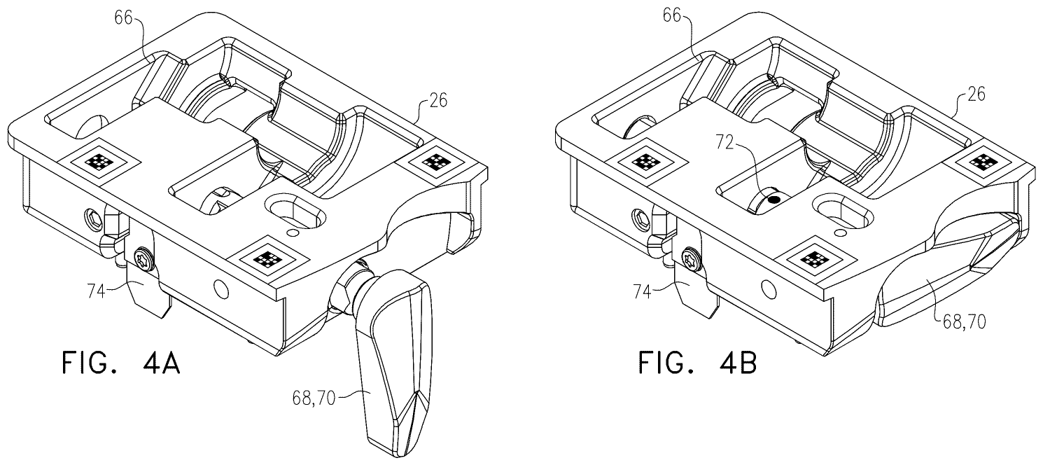

Reference is now made to FIGS. 4A-4B, which are schematic illustrations of the portion of end effector 26 to which the tool holder is coupled, in accordance with some applications of the present disclosure.

Typically, end effector 26 comprises a lock 68 configured to lock the tool holder to the end effector while the tool holder is coupled to the end effector. In particular, lock 68 is configured to transition between an unlocked state, shown in FIG. 4A, in which the lock does not lock the tool holder to the end effector, and a locked state, shown in FIG. 4B, in which the lock locks the tool holder to the end effector.

In some applications, lock 68 comprises an arm 70 configured to lock the tool holder to the end effector by rotating after passing through the tool holder. For example, in some applications, the tool holder is inserted into cavity 66 while arm 70 is in the unlocked state. Subsequently, the arm is pushed through the tool holder and rotated, such that the arm anchors the tool holder within cavity 66. In some such applications, arm 70 remains locked by virtue of frictional forces between the arm and the tool holder and/or between the arm and other portions of the end effector. To release the lock, the arm is rotated in the opposite direction with a force that is sufficient to overcome the frictional forces. Optionally, a spring (not shown) then pushes the arm out of the tool holder, such that the arm returns to the unlocked state, and the tool holder can be decoupled from the end effector.

In some applications, lock 68 comprises a magnet 72 that produces a magnetic field. End effector 26 comprises a magnetic sensor 74 configured to output a signal indicative of changes in the magnetic field, as sensed by magnetic sensor 74, resulting from the lock transitioning between the locked state and the unlocked state. For example, for applications in which lock 68 comprises arm 70 as described above, the position and orientation of magnet 72 changes as the arm is pushed and rotated, such that the power and/or direction of the magnetic field sensed by sensor 74 changes.

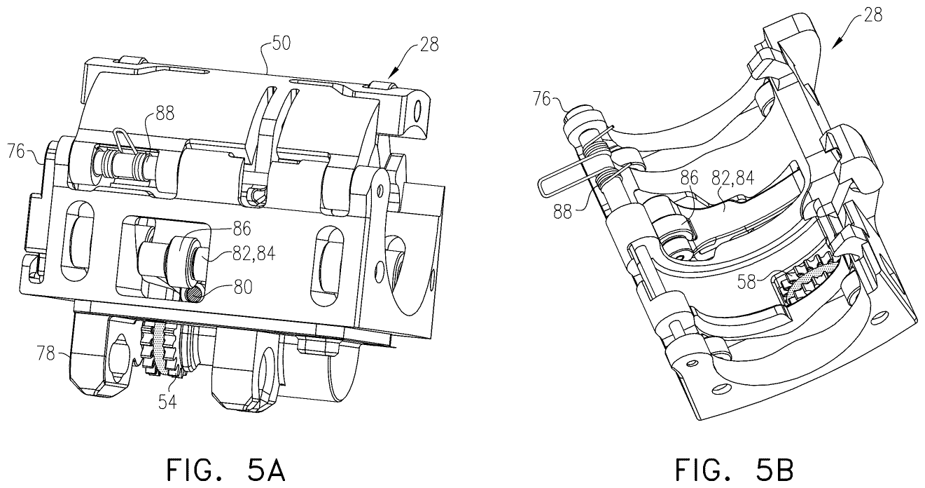

Reference is now made to FIG. 5A, which is a schematic illustration of tool holder 28, in accordance with some applications of the present disclosure. Reference is also made to FIG. 5B, which is a schematic illustration of tool holder 28 with cover 50 hidden from view, in accordance with some applications of the present disclosure.

Typically, tool holder 28 comprises a magnet 80 that produces a magnetic field, and the signal from sensor 74 (FIGS. 4A-4B) is indicative of this magnetic field. Typically, processor 40 (FIG. 1) is configured to receive the signal and to process the signal so as to ascertain whether the tool holder is coupled to the end effector. In addition, typically, the processor is configured to process the signal so as to ascertain whether the lock is in the locked state. Typically, if the tool holder is coupled to, but not locked to, the end effector, the processor displays an alert on display 44 (FIG. 1). Alternatively or additionally, if the tool holder is not locked to the end effector, the processor refrains from controlling the robotic arm to which the end effector is mounted.

In some applications, the tool holder comprises a tool support 82 comprising magnet 80. Tool support 82 is configured to support the tool, at a portion of the tool referred to herein as a “circumferential portion” of the tool, while the tool holder holds the tool, and to undergo deflection at least as the tool is placed in the tool holder such that the tool support supports the tool. The deflection of the tool support changes the position and orientation of magnet 80. Thus, the magnetic field from magnet 80 does not merely indicate whether the tool holder is coupled to the end effector. Rather, the magnetic field is sensed with different properties, such as a different power and/or direction, by the magnetic sensor, depending on the deflection. In such applications, typically, by processing the signal from the magnetic sensor, the processor ascertains one or more properties of the tool, as further described below with reference to the subsequent figures.

In some applications, tool support 82 comprises an arm 84 configured to undergo deflection by pivoting. Typically, in such applications, arm 84 comprises a roller 86 configured to contact the tool while the tool holder holds the tool, and end effector 26 (FIG. 2A) is configured to rotate the tool against roller 86. For example, in some applications, roller 86 is positioned at the end of the arm that is opposite the pivot of the arm, i.e., at the deflecting end of the arm.

In some applications, arm 84 is coupled to a spring, which biases the arm toward a default (non-deflected) position that is assumed by the arm while the tool holder does not contain a tool, and which compresses or expands as the arm is deflected. In other applications, arm 84 itself is springlike. More generally, tool support 82 may comprise any type of springlike element, or any type of deflecting element coupled to a spring.

Additional components of tool holder 28 shown in FIGS. 5A-5B include first gear 54 and second gear 58, described above with reference to FIG. 3B, a bore 78, through which arm 70 (FIGS. 4A-4B) passes while the tool holder is locked to the end effector, and a spring 88, which facilitates the opening of cover 50 via handle 76.

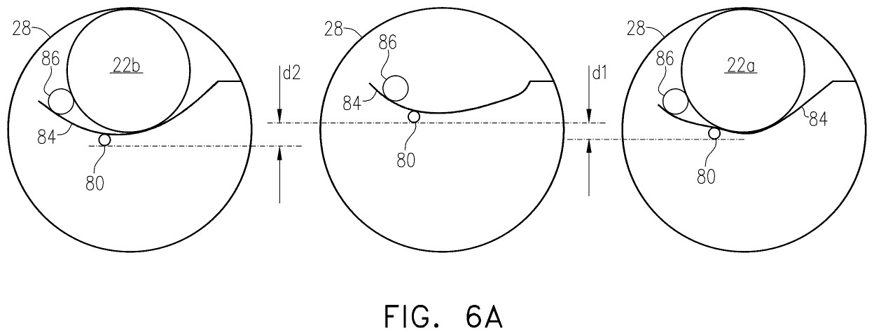

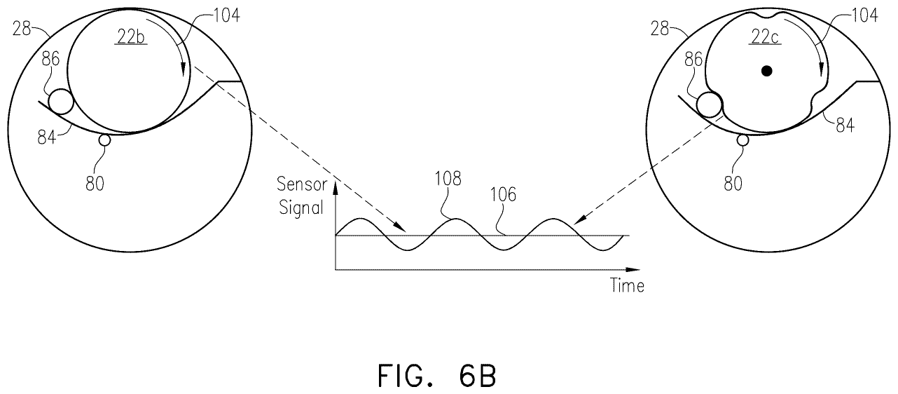

Reference is now made to FIG. 6A, which illustrates the deflection of arm 84 for two tools of different types, in accordance with some applications of the present disclosure. (In FIG. 6A, for case of illustration, tool holder 28 is depicted as a circle.)

FIG. 6A shows a cross-section through the respective circumferential portions of a first tool 22a and a second tool 22b, which are assumed to be of different types. In some applications, the respective radii of the circumferential portions of tools of different types differ from each other at at least one range of angles. For example, in some applications, as shown in FIG. 6A, the respective radii differ from each other over the entirety of the circumferential portions. The differing radii may be constant, as in FIG. 6A, or non-constant, as described below with reference to FIG. 6B.

As described above with reference to FIGS. 5A-5B, tool support 82, which in some applications comprises arm 84, is configured to support any of the tools at the circumferential portion of the tool while the tool holder holds the tool, and to undergo deflection at least as the tool is placed in the tool holder. As further described above, magnet 80 produces a magnetic field that varies (relative to the magnetic sensor) with the deflection. Thus, given that the deflection is a function of the radius of the circumferential portion of the tool, the magnetic field is indicative of the type of the tool.

For example, FIG. 6A shows that by virtue of the deflection of the arm caused by first tool 22a, magnet 80 is displaced vertically by a distance d1. In contrast, by virtue of the deflection of the arm caused by second tool 22b, magnet 80 is displaced vertically by a larger distance d2. (For simplicity, differences in the horizontal displacement and orientation of the magnet, which are typically smaller than the difference in the vertical displacement, are not explicitly indicated.) The different displacements of the magnet cause differences in the magnetic field properties, such as power and/or direction, detected by sensor 74 (FIGS. 4A-4B). Thus, by processing the signal from sensor 74, the processor can ascertain the type of the tool that is held by the tool holder.

For another example, reference is now made to FIG. 6B, which likewise illustrates the deflection of arm 84 for two tools of different types, in accordance with some applications of the present disclosure.

By way of introduction, it is noted that in the context of the present application, including the claims, the radius of a non-circular circumferential portion—which radius, by definition, is non-constant—refers to the distance between the axis of rotation 90 of the tool and the edge of the circumferential portion.

In some applications, as shown in FIG. 6B for a third tool 22c, the radius of the circumferential portion of at least one of the tools is non-constant, i.e., the circumferential portion is non-circular. By virtue of the non-constant radius, tool support 82 (e.g., arm 84) undergoes deflection as the tool rotates with respect to the tool holder. For example, for applications in which the tool rotates against roller 86, the deflection of arm 84 is a function of the radius at the point at which the circumferential portion contacts the roller, referred to below as the “contact radius.”

In some such applications, the respective radii of the circumferential portions of the tools differ from each other such that each of tools produces a unique pattern in the deflection of arm 84, relative to others of the tools, when rotated by the end effector. For example, FIG. 6B shows the rotation of second tool 22b, as indicated by a rotation indicator 104, producing a constant deflection pattern, and hence a constant signal 106 from the magnetic sensor. In contrast, the rotation of third tool 22c produces a fluctuating deflection pattern, and hence a fluctuating sensor signal 108. Thus, by processing the signal from the magnetic sensor, the processor can ascertain the type of the tool that is held by the tool holder.

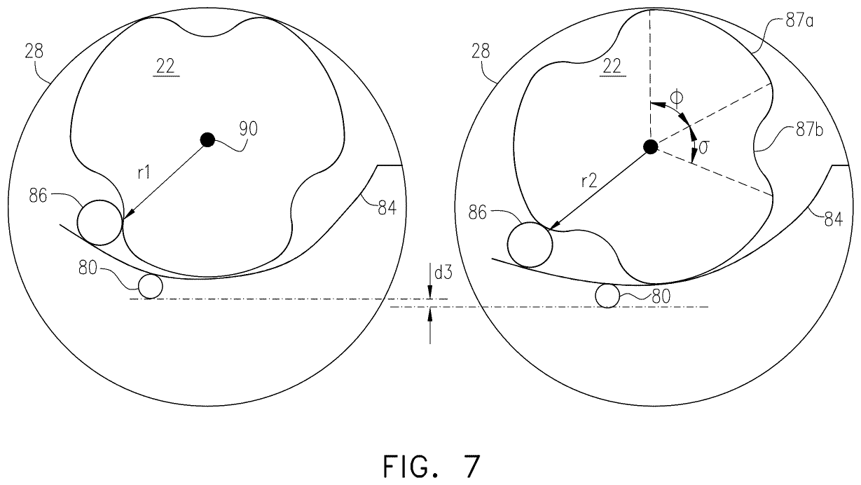

Reference is now made to FIG. 7, which is a schematic illustration of a circumferential portion of a tool 22, in accordance with some applications of the present disclosure.

As described above with reference to FIG. 6B, in some applications, the radius of the circumferential portion of at least one of the tools is non-constant, e.g., such that the deflection of arm 84 is a function of the contact radius at roller 86. For example, by virtue of the greater contact radius r2 for the orientation of the tool shown at the right of FIG. 7, relative to the contact radius r1 for the orientation shown at the left of FIG. 7, the deflection for the former orientation is greater by an amount d3, relative to the latter orientation.

Typically, in such applications, the processor is configured to process the signal from sensor 74 so as to track the rotation of the tool, i.e., to track, to some degree of precision, the number of degrees by which the tool is rotated from its initial orientation. For example, the tool shown in FIG. 7 includes three portions 87a of angular size ϕ at which the radius of the tool is r2, which alternate with three portions 87b of angular size σ at which the radius varies between values that are less than r2, such as r1. As the tool is rotated clockwise or counterclockwise, these portions facilitate calculating the number of degrees by which the tool is rotated. For example, in response to the contact radius increasing to r2 and then decreasing below r2, the processor may ascertain that the tool was rotated by ϕ degrees. In response to the contact radius then varying between values less than r2, the processor may ascertain the number of degrees, up to σ degrees, by which the tool was rotated.

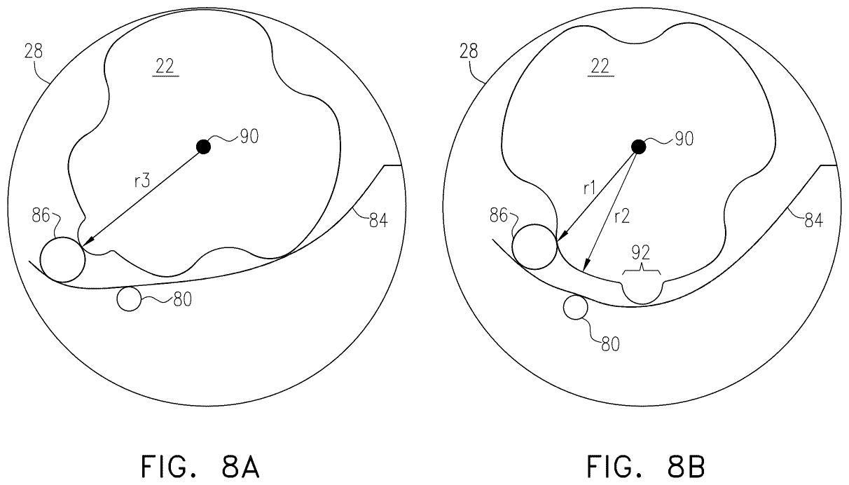

In some applications, the processor further tracks the roll orientation of the tool, i.e., the processor ascertains the initial orientation of the tool in addition to tracking the subsequent rotation of the tool. In this regard, reference is now made to FIGS. 8A-8B, which schematically illustrate the rotation of a tool 22, in accordance with some applications of the present disclosure.

In some applications, the radius of the circumferential portion of at least one of the tools at one range 92 of angles is different from (i.e., is smaller or larger than) the radius at all angles outside range 92. In some such applications, the processor uses range 92 to facilitate tracking the roll orientation of the tool. In particular, prior to using the tool to operate on the patient, the processor drives end effector 26 (FIG. 2A) to rotate the tool to a reference orientation at which the deflection of the tool support is different from at any other orientation of the tool, by virtue of the different radius. For example, in some applications, as shown in FIG. 8A, the processor drives the end effector to rotate the tool until the portion of the tool at range 92 contacts roller 86, thus attaining a contact radius r3 that is different from the contact radius at any other roll orientation of the tool. Subsequently, the processor tracks the roll orientation of the tool by tracking the rotation of the tool with respect to the reference orientation.

For example, as the tool is rotated counterclockwise from the reference orientation to the other orientation shown in FIG. 8B, the contact radius decreases from r3 to r2, and then decreases again to r1. The deflection of arm 84 changes accordingly. Thus, based on the change in the magnetic field produced by magnet 80, as sensed by the magnetic sensor, the processor can ascertain the angular size of the rotation, and thus, the orientation of the tool.

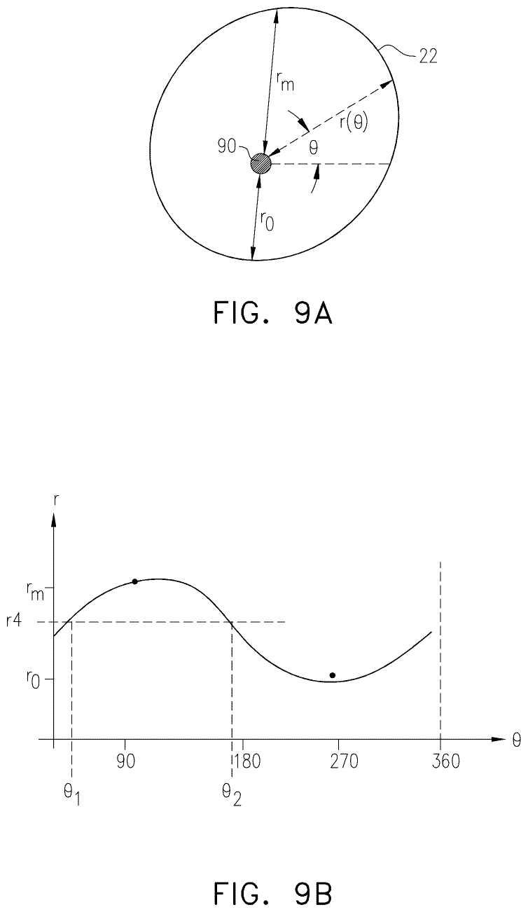

Reference is now made to FIG. 9A, which is a schematic illustration of the circumferential portion of a tool 22, in accordance with some applications of the present disclosure. Reference is also made to FIG. 9B, which plots the radius of the tool shown in FIG. 9A as a function of an angle θ, in accordance with some applications of the present disclosure.

In some applications, the radius r(θ) of the circumferential portion of the tool, where θ is the angle between an arbitrary reference point on the edge of the circumferential portion and any other point on the edge, varies continuously between a minimum value r₀ and a maximum value rₘ, each of which is attained at a single respective angle. In some such applications, the processor is configured to track the roll orientation of the tool based on the deflection of the tool support and on the change in the deflection as the tool is rotated.

For example, it will be supposed that the processor ascertains, based on the deflection of the tool support, that the contact radius is r4. Given that the radius of the tool attains the value r4 at both a first angle θ₁ and a second angle θ₂, the contact radius alone is not sufficient for ascertaining the orientation of the tool. However, as the tool rotates, the change in the deflection resolves this ambiguity. For example, it will be supposed that the tool is rotated clockwise, such that a more counterclockwise portion of the tool contacts roller 86 (FIGS. 8A-8B). In this case, the processor may ascertain that the orientation corresponds to θ₁ (i.e., that the orientation is such that the portion of the tool at θ₁ contacts the roller) in response to an increase in the deflection, which indicates an increase in the contact radius. Conversely, in response to a decrease in the deflection, which indicates a decrease in the contact radius, the processor may ascertain that the orientation corresponds to θ₂.

Thus, the application of FIG. 9A is similar to the application of FIGS. 8A-8B, in that the processor can track not only changes in the orientation of the tool, but also the orientation itself. An advantage of the application of FIG. 9A, however, is that there is no need to rotate the tool to a reference orientation prior to using the tool.

It will be appreciated by persons skilled in the art that the present disclosure is not limited to what has been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description.

1. An apparatus for operating a tool, the apparatus being for use with a robotic arm and comprising:
a tool holder, configured to hold the tool; and
an end effector, configured to mount onto the robotic arm and to couple to the tool holder while mounted onto the robotic arm, and comprising:
a lock, configured to transition between a locked state, in which the lock locks the tool holder to the end effector, and an unlocked state, in which the lock does not lock the tool holder to the end effector, and comprising a magnet that produces a magnetic field; and
a magnetic sensor, configured to output a signal indicative of changes in the magnetic field, as sensed by the magnetic sensor, resulting from the lock transitioning between the locked state and the unlocked state.

2. The apparatus according to claim 1, wherein the lock comprises an arm configured to lock the tool holder to the end effector by rotating after passing through the tool holder.

3-4. (canceled)

5. The apparatus according to claim 1, further comprising a processor configured to:
receive the signal, and
process the signal so as to ascertain whether the lock is in the locked state.

6. The apparatus according to claim 1,
wherein the magnet is a first magnet and the magnetic field is a first magnetic field,
wherein the tool holder comprises a second magnet that produces a second magnetic field, and
wherein the signal is indicative of the second magnetic field.

7. The apparatus according to claim 6, further comprising a processor configured to:
receive the signal, and
process the signal so as to ascertain whether the tool holder is coupled to the end effector.

8. The apparatus according to claim 7, wherein the processor is further configured to ascertain whether the lock is in the locked state, by processing the signal.

9. (canceled)

10. The apparatus according to claim 8, wherein the processor is further configured to refrain from controlling the robotic arm in response to ascertaining that the tool holder is coupled to, but not locked to, the end effector.

11. The apparatus according to claim 6, wherein the tool holder comprises a tool support comprising the second magnet and configured to:
support the tool while the tool holder holds the tool, and
undergo deflection at least as the tool is placed in the tool holder such that the tool support supports the tool, the second magnetic field varying with the deflection.

12. The apparatus according to claim 11, further comprising a processor configured to:
receive the signal, and
process the signal so as to ascertain a type of the tool.

13. The apparatus according to claim 11, wherein the tool support comprises an arm configured to undergo deflection by pivoting.

14. The apparatus according to claim 13,
wherein the arm comprises a roller configured to contact the tool while the tool holder holds the tool, and
wherein the end effector is configured to rotate the tool against the roller.

15. The apparatus according to claim 11,
wherein the end effector is configured to rotate the tool, with respect to the tool holder, while the tool holder holds the tool,
wherein the tool includes a circumferential portion having a non-constant radius, and
wherein the tool support is configured to undergo deflection, by virtue of the non-constant radius, as the end effector rotates the tool.

16. The apparatus according to claim 15, further comprising a processor configured to:
receive the signal, and
process the signal so as to track the rotation of the tool.

17. The apparatus according to claim 16, wherein the processor is configured to process the signal so as to track a roll orientation of the tool.

18-30. (canceled)

31. An apparatus for use with a magnetic sensor and an end effector mounted onto a robotic arm, the apparatus comprising:
multiple tools of different types comprising respective circumferential portions, respective radii of which differ from each other at at least one range of angles: and
a tool holder configured to hold any tool of the tools and to couple to the end effector so as to allow the end effector to rotate the tool with respect to the tool holder, and comprising a tool support configured to:
support the tool at the circumferential portion of the tool while the tool holder holds the tool, and
undergo deflection at least as the tool is placed in the tool holder such that the tool support supports the tool, the deflection being a function of the radius of the circumferential portion of the tool,
the tool support comprising a magnet that produces a magnetic field that is sensed with different properties by the magnetic sensor, depending on the deflection.

32-37. (canceled)

38. The apparatus according to claim 31, wherein the radius of the circumferential portion of at least one of the tools is non-constant, such that the tool support undergoes deflection as the at least one of the tools rotates with respect to the tool holder.

39. The apparatus according to claim 38, wherein the respective radii differ from each other such that each of tools produces a unique pattern in the deflection, relative to others of the tools, when rotated by the end effector.

40. The apparatus according to claim 38, wherein the radius of the circumferential portion of the at least one of the tools at one range of angles is different from the radius at all angles outside the range.

41. The apparatus according to claim 38, wherein the radius of the circumferential portion of the at least one of the tools varies continuously between a minimum value and a maximum value, each of which is attained at a single respective angle.

42. An apparatus for use with a magnetic sensor and an end effector mounted onto a robotic arm, the apparatus comprising:
a tool comprising a circumferential portion having a non-constant radius; and
a tool holder configured to hold the tool and to couple to the end effector so as to allow the end effector to rotate the tool with respect to the tool holder, and comprising a tool support configured to:
support the tool at the circumferential portion of the tool while the tool holder holds the tool, and
undergo deflection, by virtue of the non-constant radius, as the end effector rotates the tool with respect to the tool holder,
the tool support comprising a magnet that produces a magnetic field that is sensed with different properties by the magnetic sensor, depending on the deflection.

43-52. (canceled)
